(19) United States
(12) Patent Application Publication
(10) Pub. No.: US 2009/0011452 A1
(43) Pub. Date: Jan. 8, 2009

(54) Method of Prognosis of Mental Diseases, E.G. Autism and Cerebral Palsy

(76) Inventor: Frank Hoover, Bergen (NO)

Correspondence Address: KNOBBE MARTENS OLSON & BEAR LLP, 2040 MAIN STREET, FOURTEENTH FLOOR, IRVINE, CA, 92614, US

(21) Appl. No.: 11/817,184

(22) PCT Filed: Feb. 28, 2006
(86) PCT No.: PCT/GB06/00699

§ 371 Date: May 28, 2008

(51) Int. Cl. C12Q 1/02; C12M 1/00

(52) U.S. Cl. 435/29; 435/287.2; 702/19

(57) ABSTRACT

The invention provides a method of prognosis for applied behavioral analysis treatment of mental disease, in particular autism or cerebral palsy, in a human subject. The method includes analyzing a sample of body tissue or fluid from the subject for the presence or absence of a chemical marker indicative of the likelihood of success or failure of applied behavioral analysis treatment of the mental disease, and optionally, based on the analysis, beginning, continuing or ceasing applied behavioral analysis treatment of the subject. The sample is preferably an urine sample, and the preferred prognostic markers are: gluten derivatives, indolyl-3-acryloylglycine (IAG), serotonin uptake stimulator, β-casomorphineamides, gliadinomorphine, β-casomorphines, deltorphins, Demorphine, glutemorphine, gluten exophins, compounds of molecular weight of 687 Daltons.

This invention relates to a method for the prognostication of the outcome of and monitoring the progression of applied behavioural analysis (ABA) treatment of mental disorders, e.g. autism, especially childhood autism.

Autism is the name for a spectrum of developmental disorders that affect as many as 63 in 1000 children, at least 6 per 1000 having a severe form. Moreover there are indications that its prevalence is increasing. The causes of autism are not clearly understood and while some patients respond to one form of treatment, others do not.

The term autism as used herein extends to cover both severe and mild forms of autism, e.g. Aspergers syndrome.

Perhaps the most successful form of treatment for autism is that known as applied behavioural analysis (ABA), for example Early Intensive Behavioural Intervention (EIBI), and among the ABA treatments that are known TIPO—(The Early Intervention Program) has been especially effective. Indeed ABA is probably the most effective treatment for autism and no pharmaceutical treatment appears to work. Using behavioural training based on ABA (i.e. ABA treatment) on children as young as 3 years old about 50% reach approximately normal behaviour. The treatment tends to be most successful when started at an early age and thus early diagnosis of autism is important. However ABA treatment is time consuming both for the patient and the physician and moreover it is a prolonged and relatively unpleasant experience for both the patient and the physician. At present there is no way of knowing in advance whether an individual patient will be among those that benefit from ABA treatment.

While it has been known for some time that there exist biochemical markers for autism, e.g. chemicals which can be detected in the urine or blood of sufferers of autism, we have now surprisingly found that biochemical markers exist which can be correlated to a prognosis for ABA treatment in general and TIPO treatment in particular. These may correlate both to the likelihood of success in an ABA treatment regime which has not yet begun and to the success of a currently ongoing treatment. Detection of such biomarkers thus allows the physician to propose ABA treatment with a higher likelihood of success or to suggest cessation or avoidance of ABA treatment with a lower likelihood or no likelihood of success. Clearly this reduces undue suffering by the patient and expense to the family or society.

Besides EIBI and TIPO, other forms of ABA treatment include for example TEACHH.

Other forms of mental disease (or disorder) are responsive to ABA treatment and a prognosis of the likely success of ABA treatments for these diseases may also be obtained using the method of the invention. These mental diseases include for example cerebral palsy (the mental component thereof that is), developmental delay, learning disorder, mental retardation, conduct disorders, phobic anxieties, panic disorders, obsessive compulsive disorders, addictive behaviour (e.g. smoking), anorexia, bulimia, bipolar disorder, depression, ADHD, Tourettes syndrome, Retts syndrome, Fragile X, depression and pervasive development disease (PPD and PPD-NOS).

While some of these mental diseases have a physical component (e.g. as in cerebral palsy) that cannot be remedied by ABA treatment, even amelioration of only the mental component can significantly enhance the quality of life of the patient; however exposure to ABA treatment of a patient with a mental disease who is unlikely to respond to that treatment is clearly undesirable and ABA treatment is not routinely used with such diseases. Thus the prognosis method of the invention represents a significant improvement in the treatment of patients with mental disease.

Accordingly, viewed from one aspect, the invention provides a method of prognosis for applied behavioural analysis treatment of mental disease (e.g. autism) in a human subject, said method comprising analyzing a sample of body tissue or fluid from said subject for the presence or absence of a chemical marker indicative of the likelihood of success or failure of applied behavioural analysis treatment of said mental disease and, optionally, based on said analysis, beginning, continuing or ceasing applied behavioural analysis treatment of said subject.

Viewed from a further aspect the invention provides a method of assaying for a chemical marker indicative of the likelihood of success or failure of applied behavioural analysis treatment of a human subject, said method comprising analyzing a sample of body tissue or fluid from said subject for the presence or absence of said marker, optionally providing an indication of the content of said marker in said body tissue or fluid, and optionally providing an indication of the prognosis for ABA treatment.

Viewed from a further aspect the invention provides a method of theragnosis of a human subject having or suspected of having a mental disease, said method comprising analyzing a sample of body tissue or fluid from said subject for the presence or absence of a chemical marker indicative of the likelihood of success or failure of a course of treatment for said mental disease and, optionally, based on said analysis, beginning, continuing or ceasing said course of treatment, e.g. ABA treatment.

For prognosis in relation to treatment of autism, the subject is preferably a child. Indeed, in general for mental diseases which may be diagnosed in childhood, such as autism, cerebral palsy, ADHD, Tourettes syndrome, development abnormalities and Retts syndrome, etc., the subject is preferably a child. By child herein is meant a human aged up to 18 years, more particularly 1 to 16 years, especially 2 to 15 years, particularly 3 to 12 years.

The subject for the methods of the invention is preferably one already diagnosed as having the particular mental disease or less preferably one suspected of having the particular disease.

By body tissue or fluid is meant herein a fluid from the human body (e.g. blood, saliva or urine, or an extract or derivative thereof, e.g. serum or plasma), a tissue sample, or a surface cell sample (e.g. collected by rubbing a mucosal surface such as the lining of the mouth). The sample may be treated, e.g. with a preservative, antioxidant or enzyme inhibitor, and may be dried or absorbed onto a substrate. Preferably the sample is urine or serum, particularly urine, or a sample collected by swabbing the interior of the mouth. Thymol is particularly conveniently used as a preservative.

Since urine may be more or less diluted depending on the time of day it is sampled or on the drinking patterns of the patient, where the body fluid is urine the biomarker concentration determined is preferably normalized against the creatinine content of the urine which consequently is preferably also measured in the methods of the invention, Creatinine is commonly used as a normalizing factor for urine concentration in assays for analytes in urine and in the present invention may be measured by conventional means.

Chemical marker detection may conveniently be effected by chromatography, e.g. HPLC, typically using reversed phase gradient elution. However other diagnostic techniques for marker detection may of course be used, e.g. radioimmunoassays or enzyme immunoassays. Such other techniques are well known in the field of medical diagnostics and generally require the production of a binding partner for the analyte, e.g. an antibody, antibody fragment or construct, single chain antibody, oligonucleotide, etc. Antibodies to the analytes used in the methods of the invention may be prepared by standard antibody generation techniques, e.g. by immunization of mice or other mammals with immunogenic conjugates of the analyte and an immunogenic carrier (e.g. a foreign protein such as keyhole limpet hemocyanin). Alternatively binding partners may be found by panning against a chemical or biological library, e.g. a phage display library.

The generation of binding partners for analytes may also be effected by CAM-D, e.g. using the technique of EP-A-818744.

Our investigations have shown that there exists a wide range of markers for ABA treatment prognosis. Some of these have been considered previously to be biomarkers for autism as such, i.e. diagnostic markers. This it must be stressed is not inconsistent with their functioning also as prognostic markers; however it has not previously been suggested that there were any markers with prognostic value for ABA treatment.

The prognostic markers of particular interest according to the present invention appear to fall into seven groups:

1. gluten derivatives

2. indolyl-3-acryloylglycine (IAG)

3. indolyl-3-acrylic acid (in serum)

4. serotonin uptake stimulators

5. opioids and derivatives thereof

6. casein derivatives

7. unidentified urine components

For any given marker, the content is preferably compared against the mean value for a control pool (e.g. a set of asymptomatic individuals, of ABA treatment responders or of ABA treatment non-responders, preferably of similar age (e.g. within 2 years) and particularly preferably of the same sex, especially preferably a set of at least ten such control individuals). The mean and the standard deviation (SD) of the mean for the control pool are preferably determined in advance and supplied as a calibration tool to the user of the method of the invention. Values above the asymptomatic control mean are considered indicative of relevance, values above control mean plus SD are more indicative of relevance and values above control mean+2×SD are highly indicative of relevance. In the event of testing taking place at two or more times before, during or after ABA treatment, reduction in values, especially from above control +2×SD to below control +2×SD, is considered indicative of at least partial success, i.e. indicative that treatment should be continued or completed.

More specifically, the following chemical compounds have been found to be prognostic markers, in particular for autism:

1. The gluten derivatives which, on HPLC, elute after hippuric acid and before IAG.

2. IAG

3. Serotonin uptake stimulator (pyro-Glu-Trp-Gly-NH₂).

4. The casein derivatives that elute after the serotonin uptake stimulator and before the beta-casomorphineamides

5. The beta-casomorphineamides, e.g. beta-casomorphine-1-3-amide, beta-casomorphine-1-4-amide and beta-casomorphine-1-5-amide, especially beta-casomorphine-1-4-amide (Tyr-Pro-Phe-Pro-NH₂).

6. Gliadinomorphine

7. The beta-casomorphines, e.g. beta-casomorphine 1-3(Tyr-Pro-Phe), beta-casomorphine 1-5 (Tyr-Pro-Phe-Pro-Gly), and beta-casomorphine 1-4 (Tyr-Pro-Phe-Pro).

8. Compounds of molecular weight about 687 Daltons.

9. Deltorphins, e.g. A, I(C) or II(B)

10. Dermorphine

11. Glutemorphine

12. Gluten Exophins, e.g. A5, B5 and C.

Prognosis for poor or no response to ABA treatment may be made on the basis of an HPLC peak area (or height) which is significantly higher than the asymptomatic control mean and wherein the total peak area (or heights) for the gluten derivatives is greater than that for IAG and/or the total peak area (or heights) for the serotonin uptake stimulator is greater than that for IAG. (However, abnormally high levels of IAG may themselves be predictive of Aspergers and/or prognostic for ABA treatment therefor). A control factor that may be used is that the total peak area (or heights) for IAG should itself be at least 1.5 times, e.g. at least twice, the value for the asymptomatic control set. Moreover, the gluten derivatives show at least two peaks in the HPLC trace between the peaks for hippuric acid and for IAG. The second, the more prominent, of these peaks, may be used in prognosis as above. The first of these peaks on its own is indicative of poor or no response to ABA treatment when area (or height) is significantly higher than the values for control sets of patients responding to ABA treatment (itself higher than the equivalent value for an asymptomatic control set). Likewise the IAG peak is on its own indicative of poor prognosis for response where its height is significantly greater (e.g. at least 50% greater) than that for a control set of patients responding to ABA treatment (again itself higher than the equivalent value for an asymptomatic control set).

The detection technique used in the method of the invention may be any appropriate analytical technique. However the use of liquid chromatography is preferred, especially HPLC. In an alternative embodiment, however, a hyphenated technique, i.e. one involving a separation technique combined with a spectroscopic technique, may advantageously be used. examples of such techniques include LC and GC combined with MS or NMR, preferably LC-MS. Such hyphenated techniques have the advantage that the “result”, i.e. poor/good prospects of successful treatment or poor/good progression of existing treatment may be generated by applying a prediction matrix (generated from asymptomatic controls, responder controls and poor responder controls) to the data matrix from the hyphenated technique. Such a prediction matrix can moreover be generated without chemical identification of the biomarkers, e.g. as described in WO 02/03056. Thus viewed from a further aspect the invention provides a prediction matrix applicable to the data matrix of a hyphenated analytical technique to produce a value indicative of prognosis for response to ABA treatment of a mental disease, e.g. of autism.

The use of a prediction matrix and a hyphenated analysis technique is of particular relevance when the sample being assayed contains protein and nucleic acids, e.g. where a blood sample is used. In this event, the sample may optionally be pretreated with enzymes such as DNAases or proteases. In this way genetic markers for treatment prognostication may contribute to the prediction matrix even where they have yet to be identified. Where the prediction matrix contains a significant contribution from a nucleic acid, e.g. an oligonucleotide, the relevant chromatographic fraction may be further analysed to detect the relevant gene sequence or fragment.

Where the predictive marker is chemically identified, it may conveniently be assayed for in an automated assay device, e.g. as disclosed in WO 02/090995, using an assay cartridge containing all the required assay reagents, e.g. buffers, antibodies, etc. Thus viewed from a further aspect the invention provides an assay cartridge for use in an assay for a marker for a mental disease (e.g. autism, i.e. a diagnostic marker such as IAG) or for a prognostic marker for response to ABA treatment of that mental disease. In practice, it may be desirable to use two such cartridges when performing an assay on a urine sample, a first to determine a value for the marker and a second to determine a value for a reference compound, e.g. creatinine, whereby the marker value may be adjusted to allow for urine concentration as discussed earlier.

Viewed from a further aspect the invention provides a computer, e.g. functionally linked to a hyphenated analysis machine, programmed with the prediction matrix of the invention. Viewed from a yet further aspect the invention provides a data carrier, e.g. a chip, disc or tape, programmed with the prediction matrix of the invention.

Besides the prediction matrix based on hyphenated techniques, principal component analysis (PCA) may be used to generate a prediction or prediction matrix using hyphenated or non-hyphenated spectroscopic or chromatographic techniques effected on unknown samples and compared with known (control) samples.

The aspects of the present invention are applicable more generally than in relation to autism alone, and in particular that they are applicable to learning disorders and mental disorders in general, e.g. mental retardation, mental developmental delay, depression, schizophrenia, dyslexia and attention deficit disorders (e.g. ADHD). In such cases, the invention may be used to prognosticate the likely efficacy of behavioural treatment. It may also be used to predict when behavioural treatment should be used in preference to pharmaceutical treatment, e.g. treatment with sedatives or other mood-altering drugs such as ritalin. As mentioned above these form further aspects of the present invention.

The invention will now be described further with reference to the following non-limiting Examples and the accompanying drawings, in which:

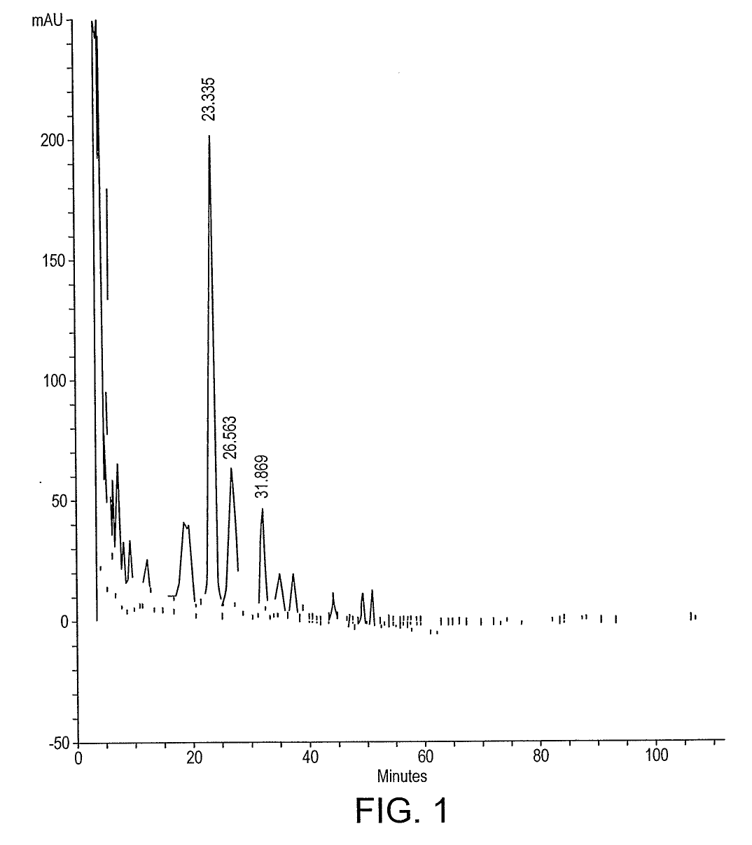

FIG. 1 is an HPLC trace for urine from an autism-asymptomatic control set;

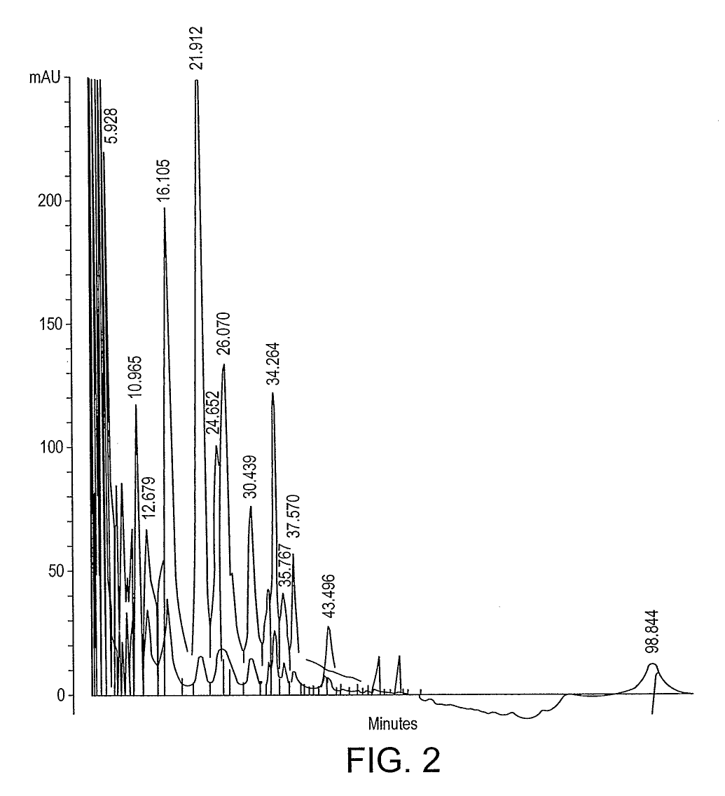

FIG. 2 is an HPLC trace for urine pooled from a set of patients having autism and responsive to ABA treatment;

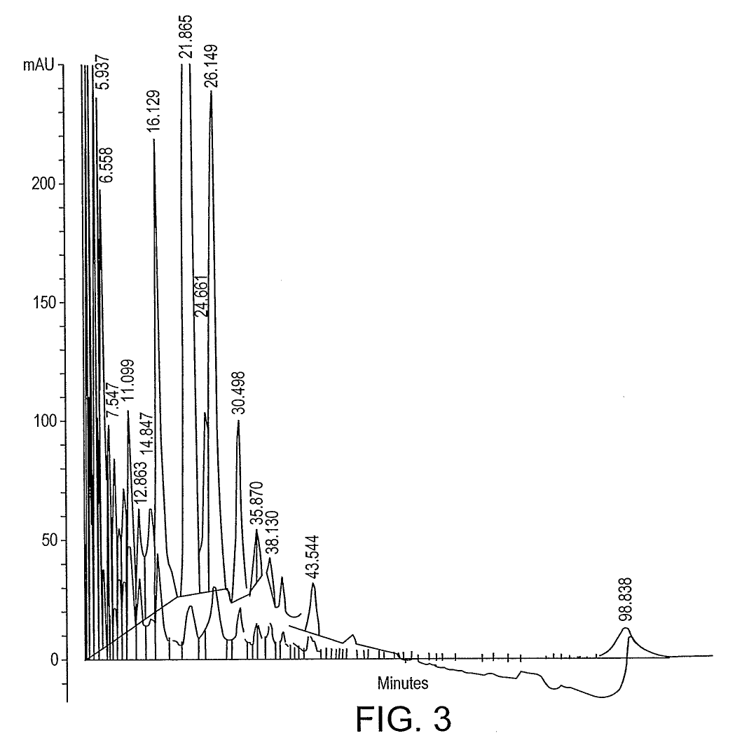

FIG. 3 is an HPLC trace for urine pooled from a set of patients having autism and not being responsive to ABA treatment;

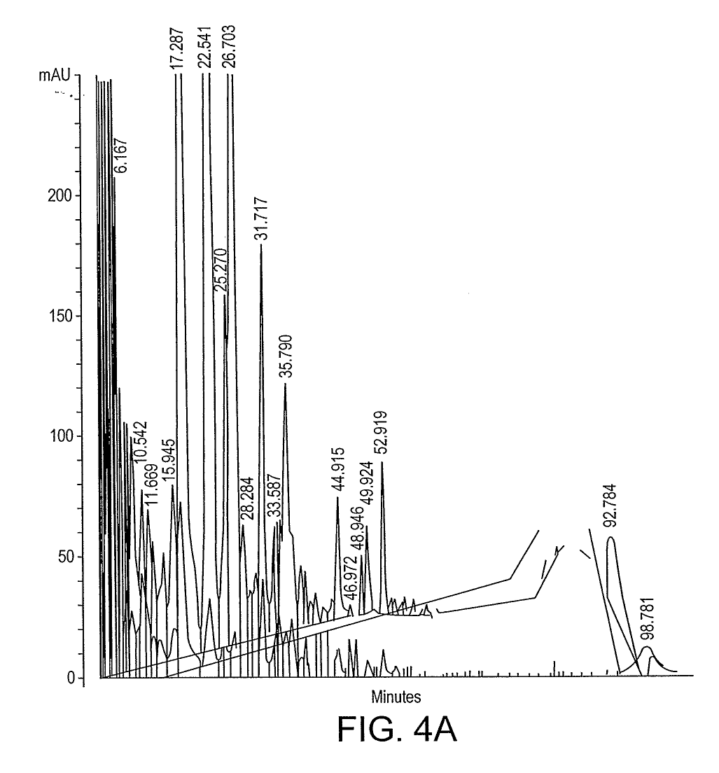

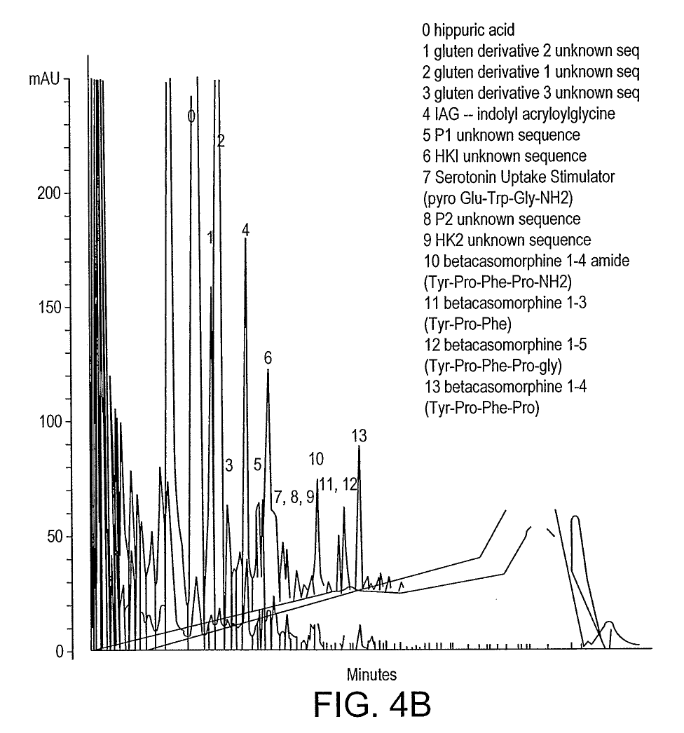

FIG. 4 is an HPLC trace for urine from a patient having autism and not being responsive to ABA treatment;

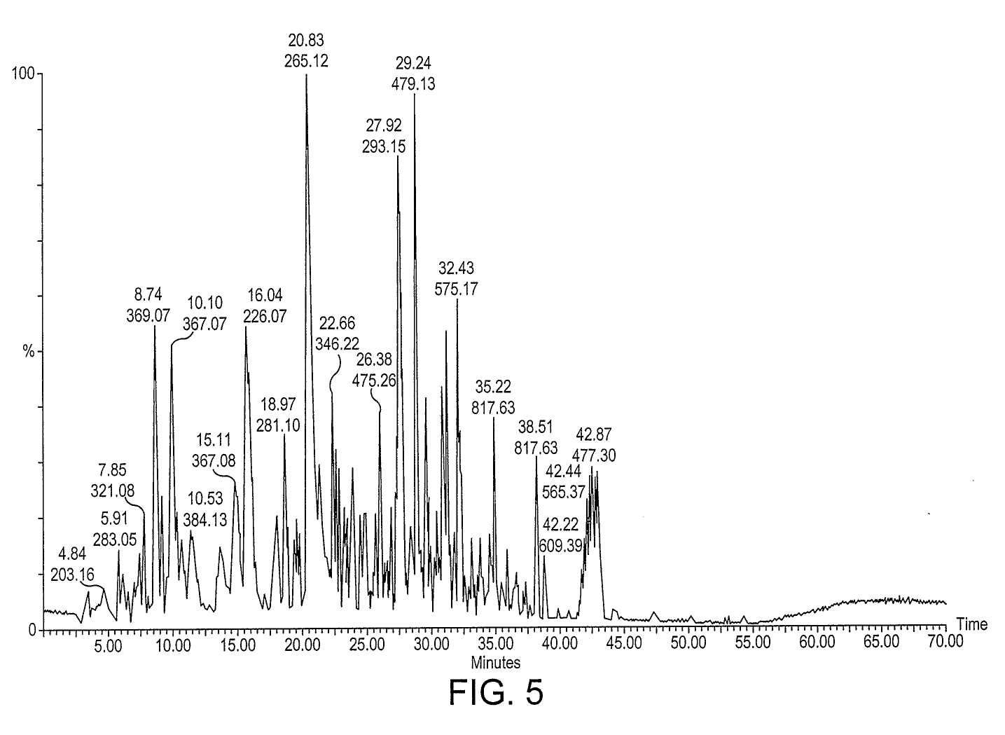

FIG. 5 is a mass spectrum trace for urine from a patient having autism and not being responsive to ABA treatment;

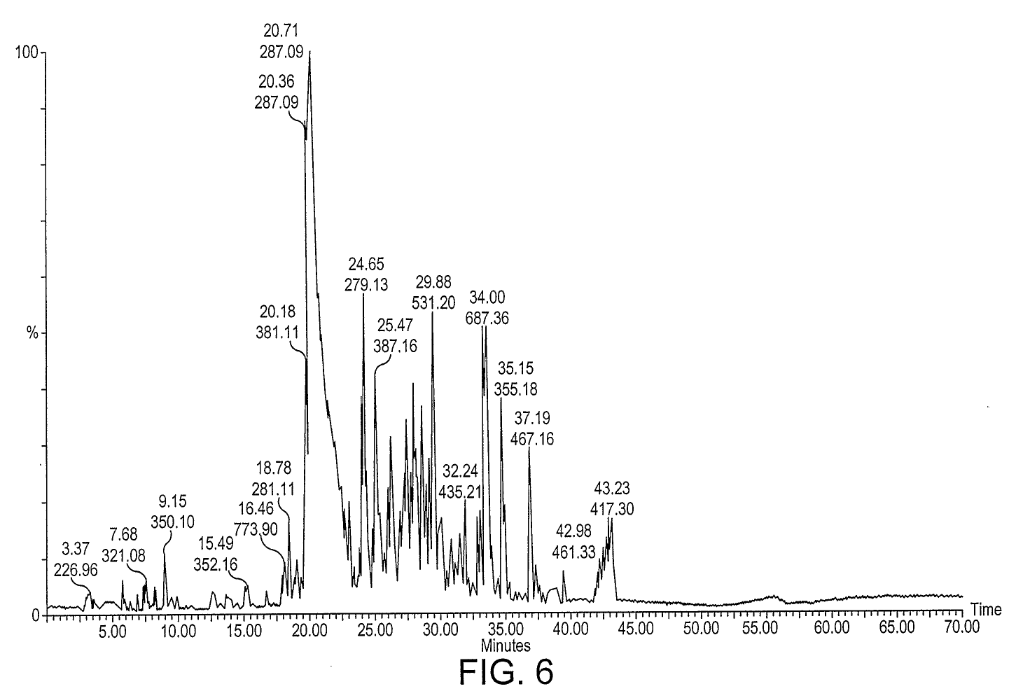

FIG. 6 is a mass spectrum trace for urine from a patient having autism and being responsive to ABA treatment;

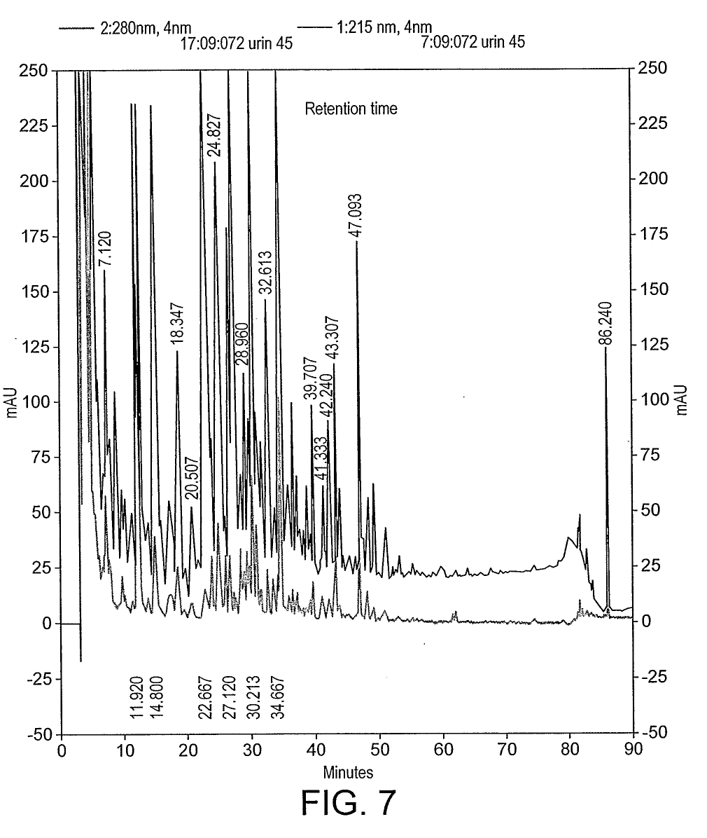

FIG. 7 is an HPLC trace for urine from a female monozygotic twin diagnosed as having cerebral palsy; and

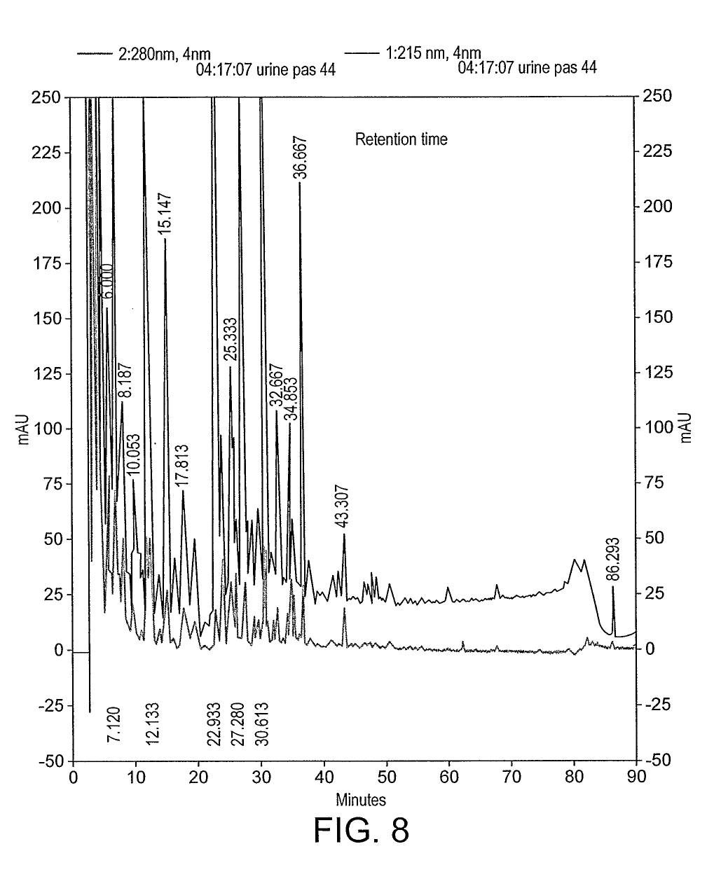

FIG. 8 is an HPLC trace for urine from the cerebral palsy-asymptomatic female twin of the twin of FIG. 7.

### EXAMPLE 1

### HPLC Examination of Urine Samples

Urine was collected as the first morning urine prior to food or drink.

Urine was collected in a sample container and frozen at −20° C.

Urine was thawed, spun in a centrifuge for 10 minutes at ambient temperature at 4000×g, measured for pH and filtered through a Costar Spin X cellulose acetate filter.

Urine was measured for creatinine content by standard clinical procedures.

Urine containing 250 nm of creatinine was applied to the HPLC.

Urine was passed through a reverse phase c18 column in trifluoroacetic acid with an acetonitrile gradient.

Acetonitrile concentration was 10 mM in trifluoracetic acid:

Gradient was as follows:

| Time | % Acetonitrile |
|---|---|
| 15 | 0 |
| 75 | 40 |
| 80 | 60 |
| 89 | 60 |
| 94 | 0 |
| 115 | 0 |

### EXAMPLE 2

### HPLC Results

Peaks are very consistent, they appear in sequence with little deviation after the main peak (Hippuric Acid-HA) which typically elutes at 22-23 min. Thus all molecules afterward come out in sequence. The system is manipulated with buffer concentration so that HA does come out at this time point allowing the assignment of the other molecules. Controls are run with some of the purified molecules on each run to test where they elute.

Peaks of interest are following HA (peak 0, see FIG. 4 labeled for ID). Peak elution times are in parentheses.

Of note in the non-responder pool is at least peaks 1 (24.6), 2(26.1), 4(30.4), 6(33.7), 10(43.5)

Of note in the responder pool is at least peaks 6 (34.2), 7 (35.7), 8(37.5), 10(43.4)

The labeled FIG. 4 shows additional peaks, and this patient does not express the full spectrum of described peaks. Not every patient has the same profile. Other peaks which appear to be important are beta casomorphin 1-6 (, beta casomorphin 1-7 (61 m), beta casomorphin 1-8 (63 m), Deltorphin (A, 56 min). Deltorphin 1 or (C), Deltorphin II or (B), Dermorphin (59 min), A4 Glutemorphin (42 m), Gluten Exophrin A5 (43 m), Gluten Exorphin B5, Gluten Exorphin C, Gliadinomorphin (51-52 min), beta casomorphine 1-5 amide (65 m). Relative elution times are in parentheses.

### EXAMPLE 3

### Mass Spectra

A capillary system was used with a C18 reverse phase column into a TOF-MS scanned from 200-2000. One non responder and one responder are provided. The samples were simply run and not normalized for creatinine. Of note, there are several differences in the profiles from non-responder to responder, the most obvious being a peak of MW at 687.36 at 34 minutes in the responder. Inspection revealed that this peak was not present in the 3 non-responders that were examined. Inspection of the HPLC chromatograms from this patient and the others, revealed no major differences in the absolute expression of compounds.

### EXAMPLE 4

### Cerebral Palsy

Urine samples from two monozygotic female twins, one having cerebral palsy and one free from cerebral palsy were taken and subjected to HPLC analysis as in Example 1. The HPLC trace for the twin with cerebral palsy and the twin without cerebral palsy are shown in FIGS. 7 and 8 respectively. It may readily be seen that the trace for the twin with cerebral palsy contains many peaks that are either absent from or less prominent in the HPLC trace for the twin without cerebral palsy.

It was found that differences were repeatable and that the compounds they relate to are apparently stable through relatively long incubation (>8 hours) at ambient temperature and through repeated freeze/thaw cycles. Examples of HPLC peaks present significantly in the affected twin but absent or less prominent in the non-affected twin include those at 11.920, 14.800, 22.667, 27.120, 30.213, 34.667, 39.707, 41.333, 42.240, 47.093 and 86.240 minutes. These figures may of course be rounded up to one or two decimal points.

1. A method of prognosis for applied behavioral analysis treatment of mental disease in a human subject, said method comprising analyzing a sample of body tissue or fluid from said subject for the presence or absence of a chemical marker indicative of the likelihood of success or failure of applied behavioral analysis treatment of said mental disease.

2. A method of assaying for a chemical marker indicative of the likelihood of success or failure of applied behavioral analysis treatment of a human subject, said method comprising analyzing a sample of body tissue or fluid from said subject for the presence or absence of said marker.

3. A method of theragnosis of a human subject having or suspected of having a mental disease, said method comprising analyzing a sample of body tissue or fluid from said subject for the presence or absence of a chemical marker indicative of the likelihood of success or failure of a course of treatment for said mental disease.

4. The method as claimed in any one of claims 1 to 3 wherein said mental disease is autism.

5. The method as claimed in any one of claims 1 to 3 wherein said mental disease is cerebral palsy.

6. The method as claimed in any one of claims 1 to 3 wherein said subject has already been diagnosed as having said mental disease.

7. The method as claimed in any one of claims 1 to 3 wherein said subject is a child.

8. The method as claimed in any one of claims 1 to 3 wherein said sample is a urine sample.

9. A prediction matrix applicable to the data matrix of a hyphenated analytical technique to produce a value indicative of prognosis for response to applied behavioral analysis (ABA) treatment of a mental disease.

10. An assay cartridge for use in an assay for a marker for a mental disease or for a prognostic marker for response to applied behavioral analysis (ABA) treatment of a mental disease.

11. The method of claim 1, further comprising, based on said analysis, beginning, continuing or ceasing applied behavioral analysis treatment of said subject.

12. The method of claim 2, further comprising providing an indication of the content of said marker in said body tissue or fluid.

13. The method of claim 12, further comprising providing an indication of the prognosis for applied behavioral analysis (ABA) treatment.

14. The method of claim 3, further comprising, based on said analysis, beginning, continuing or ceasing said course of treatment.
